(19) United States
(12) Patent Application Publication
(10) Pub. No.: US 2022/0257570 A1
(43) Pub. Date: Aug. 18, 2022

(54) CALPAIN INHIBITORS AND USES THEREOF FOR TREATING NEUROLOGICAL DISORDERS

(71) Applicant: Blade Therapeutics, Inc., South San Francisco, CA (US)

(72) Inventors: Prabha Ibrahim, Mountain View, CA (US); Maria Fuentes, San Francisco, CA (US); P. T. Ravi Rajagopalan, San Ramon, CA (US); Walter Yu, South San Francisco, CA (US)

(21) Appl. No.: 17/629,988

(22) PCT Filed: Jul. 28, 2020
(86) PCT No.: PCT/US2020/043888

§ 371 Date: Jan. 25, 2022

Related U.S. Application Data
(60) Provisional application No. 62/881,274, filed on Jul. 31, 2019.

(51) Int. Cl. A61K 31/433; A61P 25/28; A61K 9/20; A61K 9/48; A61K 9/10; A61K 9/00; A61K 31/166; A61K 31/415; A61K 31/343; A61K 31/138; A61K 31/135; A61K 31/554; A61K 31/4545; A61K 31/451; A61K 31/5415; A61K 31/19; A61K 31/55; A61K 31/53

(52) U.S. Cl. CPC A61K 31/433; A61K 31/53; A61K 9/2059; A61K 9/2054; A61K 9/2013; A61K 9/2018; A61K 9/4858; A61K 9/10; A61K 9/0019; A61K 31/166; A61K 31/415; A61K 31/343; A61K 31/138; A61K 31/135; A61K 31/554; A61K 31/4545; A61K 31/451; A61K 31/5415; A61K 31/19; A61K 31/55; A61P 25/28

(57) ABSTRACT

Methods of treating neurological diseases and disorders associated with protein aggregation using calpain inhibitors are provided. Said neurological diseases associated with protein aggregation include polyglutamine expansion diseases such as Huntington's disease, Machado-Joseph disease, and spinocerebellar ataxias.

### BACKGROUND

### Field

The present application relates to the fields of pharmaceutical chemistry, biochemistry and medicine. More particularly, the present disclosure relates to calpain inhibitors and their use as therapeutic agents.

### Description of the Related Art

Polyglutamine (PolyQ)-related disorders are genetic disorders manifested by progressive neurodegeneration resulting in behavioral and physical impairments. PolyQ-containing proteins are ubiquitously expressed throughout the body; however the pathology is primarily restricted to neuronal tissue. Calpain inhibition can potentially be beneficial in PolyQ disorders.

Huntington's disease is an inherited, progressive neurodegenerative disorder characterized by choreiform movements, psychiatric problems, and dementia. Huntington's disease is caused by an expanded cytosine-adenine-guanine (CAG) repeat length in the Huntingtin (HTT) gene, causing an expanded polyglutamine tract in the huntingtin protien and resulting in accumulation of mutant huntingtin protein in the brain, leading to disrupted postsynaptic signaling. The greater the number of CAG repeats, the earlier the age of onset and greater severity of disease. Complications typically cause death 10-30 years after onset.

The spinocerebellar ataxias (SCAs) are a large family of genetically and clinically heterogenous disorders caused by expanded CAG repeats. Machado-Joseph disease (MJD), or spinocerebellar ataxia 3 (SCA3), is the most common SCA and is characterized by a slow degeneration of the cerebellum, which leads to ataxia and cognitive impairments. MJD is caused by an expanded CAG repeat length in the MJD gene, which codes for ataxin 3. This mutation causes the formation of neuronal inclusions and subsequent neurodegeneration. Most patients require a wheelchair 10-15 years after onset.

The current standard of care for PolyQ-related disorders entails symptomatic treatment with antipsychotics and dopamine-depleting agents along with supportive care. Unfortunately, there are no currently available disease modifying therapies for PolyQ-related disorders.

### SUMMARY

In some embodiments, provided herein is a method of treating a neurological disease or disorder associated with protein aggregation, the method comprising administering to a subject in need thereof a compound of Formula (I):

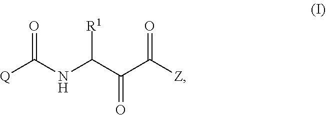

or a pharmaceutically acceptable salt or prodrug thereof, wherein:

R¹ may be —C₁₋₆alkyl or —(CH₂)ₙ—C₆₋₁₀aryl optionally substituted with one, two, or three substituents independently selected from the group consisting of: -halo, —C₁₋₆ alkyl, —C₁₋₆haloalkyl, —C₁₋₆alkoxy, and —C₁₋₆haloalkoxy;

Z is —NR²R³ or —OR⁴;

R² is -hydrogen or —C₁₋₆ alkyl;

R³ is -hydrogen, —C₁₋₆ alkyl, —C₃₋₁₀ cycloalkyl, or —OR⁴;

R⁴ is -hydrogen or —C₁₋₆ alkyl;

Q is -5-10-membered heteroaryl optionally substituted with one, two, or three substituents independently selected from the group consisting of: -halo; —C₁₋₆ alkyl; —C₁₋₆haloalkyl; —C₁₋₆ alkoxy; —C₁₋₆ haloalkoxy; and —C₆₋₁₀ aryl optionally substituted with one, two, or three substituents independently selected from the group consisting of: -halo, —C₁₋₆ alkyl, —C₁₋₆ haloalkyl, —C₁₋₆alkoxy, and —C₁₋₆haloalkoxy; or

Q is —C₆₋₁₀aryl optionally substituted with one, two, or three substituents independently selected from the group consisting of: -halo; —C₁₋₆ alkyl; —C₁₋₆ haloalkyl; —C₁₋₆alkoxy; —C₁₋₆haloalkoxy; and -5-10-membered heteroaryl optionally substituted with one, two, or three substituents independently selected from the group consisting of: -halo, —C₁₋₆ alkyl, —C₁₋₆ haloalkyl, —C₁₋₆alkoxy, and —C₁₋₆haloalkoxy; and

n is 1 or 2.

In some embodiments, the method may further comprise administering to the subject one or more second pharmaceutical agents. In some embodiments, the second pharmaceutical agent may be selected from tetrabenazinem, deutetrabenazine, citalopram, escitalipram, fluoxetine, sertraline, quetiapine, risperidone, haloperidol, chlorpromazine, valproate, carbamazepine, lamotrigine, levodopa, baclofen, and botulinum toxin.

In some embodiments, the neurological disorder associated with protein aggregation may be a polyglutamine disease or disorder. In some specific embodiments, the polyglutamine disorder may be Huntington's disease, Machado-Joseph disease, dentatorubral-pallidoluysian atrophy, spinal and bulbar muscular atrophy, spinocerebellar ataxia type 1 (SCA1), spinocerebellar ataxia type 2 (SCA2), spinocerebellar ataxia type 6 (SCA6), spinocerebellar ataxia type 7 (SCA7), or spinocerebellar ataxia type 17 (SCA17). In some embodiments, the neurological disorder associated with protein aggregation may be Alzheimer's disease, Parkinson's disease, or amyotrophic lateral sclerosis.

### DETAILED DESCRIPTION OF THE PREFERRED EMBODIMENT

### Definitions

Unless defined otherwise, all technical and scientific terms used herein have the same meaning as is commonly understood by one of ordinary skill in the art to which this disclosure belongs. All patents, applications, published applications, and other publications are incorporated by reference in their entirety. In the event that there is a plurality of definitions for a term herein, those in this section prevail unless stated otherwise.

“Subject” as used herein, means a human or a non-human mammal, e.g., a dog, a cat, a mouse, a rat, a cow, a sheep, a pig, a goat, a non-human primate or a bird, e.g., a chicken, as well as any other vertebrate or invertebrate.

The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rats and mice but also includes many other species.

An “effective amount” or a “therapeutically effective amount” as used herein refers to an amount of a therapeutic agent that is effective to relieve, to some extent, or to reduce the likelihood of onset of, one or more of the symptoms of a disease or condition, and includes curing a disease or condition. “Curing” means that the symptoms of a disease or condition are eliminated; however, certain long-term or permanent effects may exist even after a cure is obtained (such as extensive tissue damage).

“Treating” or “treatment” of a disease or disorder in a subject as used herein refers to 1) preventing the disease or disorder from occurring in a subject that is predisposed or does not yet display symptoms of the disease or disorder; 2) inhibiting the disease or disorder or arresting its development; or 3) ameliorating or alleviating the cause of the regression of the disease or disorder.

As used herein, the term “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. Conventional procedures for the selection and preparation of suitable prodrug derivatives are described, for example, in Design of Prodrugs, (ed. H. Bundgaard, Elsevier, 1985), which is hereby incorporated herein by reference in its entirety.

As used herein, the term “pro-drug ester” refers to derivatives of the compounds disclosed herein formed by the addition of any of several ester-forming groups that are hydrolyzed under physiological conditions. Examples of pro-drug ester groups include pivoyloxymethyl, acetoxymethyl, phthalidyl, indanyl and methoxymethyl, as well as other such groups known in the art, including a (5-R-2-oxo-1,3-dioxolen-4-yl)methyl group. Other examples of pro-drug ester groups can be found in, for example, T. Higuchi and V. Stella, in “Pro-drugs as Novel Delivery Systems”, Vol. 14, A.C.S. Symposium Series, American Chemical Society (1975); and “Bioreversible Carriers in Drug Design: Theory and Application”, edited by E. B. Roche, Pergamon Press: New York, 14-21 (1987) (providing examples of esters useful as prodrugs for compounds containing carboxyl groups). Each of the above-mentioned references is herein incorporated by reference in their entirety.

“Metabolites” of the compounds disclosed herein include active species that are produced upon introduction of the compounds into the biological milieu.

“Solvate” refers to the compound formed by the interaction of a solvent and a compound described herein, a metabolite, or salt thereof. Suitable solvates are pharmaceutically acceptable solvates including hydrates.

The term “pharmaceutically acceptable salt” refers to salts that retain the biological effectiveness and properties of a compound, which are not biologically or otherwise undesirable for use in a pharmaceutical. In many cases, the compounds herein are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in WO 87/05297, Johnston et al., published Sep. 11, 1987 (incorporated by reference herein in its entirety).

As used herein, “Cₐ to C_{b}” or “C_{a-b}” in which “a” and “b” are integers refer to the number of carbon atoms in the specified group. That is, the group can contain from “a” to “b”, inclusive, carbon atoms. Thus, for example, a “C₁ to C₄ alkyl” or “C₁₋₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—.

The term “halogen” or “halo,” as used herein, means any one of the radio-stable atoms of column 7 of the Periodic Table of the Elements, e.g., fluorine, chlorine, bromine, or iodine, with fluorine and chlorine being preferred.

As used herein, “alkyl” refers to a straight or branched hydrocarbon chain that is fully saturated (i.e., contains no double or triple bonds). The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 4 carbon atoms. The alkyl group of the compounds may be designated as “C₁₋₄ alkyl” or similar designations. By way of example only, “C₁₋₄ alkyl” indicates that there are one to four carbon atoms in the alkyl chain, i.e., the alkyl chain is selected from the group consisting of methyl, ethyl, propyl, isopropyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

As used herein, “haloalkyl” refers to a straight- or branched-chain alkyl group having from 1 to 12 carbon atoms in the chain, substituting one or more hydrogens with halogens. Examples of haloalkyl groups include, but are not limited to, —CF₃, —CHF₂, —CH₂F, —CH₂CF₃, —CH₂CHF₂, —CH₂CH₂F, —CH₂CH₂Cl, —CH₂CF₂CF₃ and other groups that in light of the ordinary skill in the art and the teachings provided herein, would be considered equivalent to any one of the foregoing examples.

As used herein, “alkoxy” refers to the formula —OR wherein R is an alkyl as is defined above, such as “C₁₋₉ alkoxy”, including but not limited to methoxy, ethoxy, n-propoxy, 1-methylethoxy (isopropoxy), n-butoxy, iso-butoxy, sec-butoxy, and tert-butoxy, and the like.

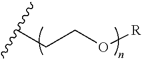

As used herein, “polyethylene glycol” refers to the formula wherein n is an integer greater than one and R is a hydrogen or alkyl. The number of repeat units “n” may be indicated by referring to a number of members. Thus, for example, “2- to 5-membered polyethylene glycol” refers to n being an integer selected from two to five. In some embodiments, R is selected from methoxy, ethoxy, n-propoxy, 1-methylethoxy (isopropoxy), n-butoxy, iso-butoxy, sec-butoxy, and tert-butoxy.

As used herein, “heteroalkyl” refers to a straight or branched hydrocarbon chain containing one or more heteroatoms, that is, an element other than carbon, including but not limited to, nitrogen, oxygen and sulfur, in the chain backbone. The heteroalkyl group may have 1 to 20 carbon atoms although the present definition also covers the occurrence of the term “heteroalkyl” where no numerical range is designated. The heteroalkyl group may also be a medium size heteroalkyl having 1 to 9 carbon atoms. The heteroalkyl group could also be a lower heteroalkyl having 1 to 4 carbon atoms. In various embodiments, the heteroalkyl may have from 1 to 4 heteroatoms, from 1 to 3 heteroatoms, 1 or 2 heteroatoms, or 1 heteroatom. The heteroalkyl group of the compounds may be designated as “C₁₋₄ heteroalkyl” or similar designations. The heteroalkyl group may contain one or more heteroatoms. By way of example only, “C₁₋₄ heteroalkyl” indicates that there are one to four carbon atoms in the heteroalkyl chain and additionally one or more heteroatoms in the backbone of the chain.

The term “aromatic” refers to a ring or ring system having a conjugated pi electron system and includes both carbocyclic aromatic (e.g., phenyl) and heterocyclic aromatic groups (e.g., pyridine). The term includes monocyclic or fused-ring polycyclic (i.e., rings which share adjacent pairs of atoms) groups provided that the entire ring system is aromatic.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms, although the present definition also covers the occurrence of the term “aryl” where no numerical range is designated. In some embodiments, the aryl group has 6 to 10 carbon atoms. The aryl group may be designated as “C₆₋₁₀ aryl,” “C₆ or C₁₀ aryl,” or similar designations. Examples of aryl groups include, but are not limited to, phenyl, naphthyl, azulenyl, and anthracenyl.

As used herein, “aryloxy” and “arylthio” refers to RO— and RS—, in which R is an aryl as is defined above, such as “C₆₋₁₀ aryloxy” or “C₆₋₁₀ arylthio” and the like, including but not limited to phenyloxy.

An “aralkyl” or “arylalkyl” is an aryl group connected, as a substituent, via an alkylene group, such “C₇₋₁₄ aralkyl” and the like, including but not limited to benzyl, 2-phenylethyl, 3-phenylpropyl, and naphthylalkyl. In some cases, the alkylene group is a lower alkylene group (i.e., a C₁₋₄ alkylene group).

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members (i.e., the number of atoms making up the ring backbone, including carbon atoms and heteroatoms), although the present definition also covers the occurrence of the term “heteroaryl” where no numerical range is designated. In some embodiments, the heteroaryl group has 5 to 10 ring members or 5 to 7 ring members. The heteroaryl group may be designated as “5-7 membered heteroaryl,” “5-10 membered heteroaryl,” or similar designations. In various embodiments, a heteroaryl contains from 1 to 4 heteroatoms, from 1 to 3 heteroatoms, from 1 to 2 heteroatoms, or 1 heteroatom. For example, in various embodiments, a heteroaryl contains 1 to 4 nitrogen atoms, 1 to 3 nitrogen atoms, 1 to 2 nitrogen atoms, 2 nitrogen atoms and 1 sulfur or oxygen atom, 1 nitrogen atom and 1 sulfur or oxygen atom, or 1 sulfur or oxygen atom. Examples of heteroaryl rings include, but are not limited to, furyl, thienyl, phthalazinyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, triazinyl, quinolinyl, isoquinlinyl, benzimidazolyl, benzoxazolyl, benzothiazolyl, indolyl, isoindolyl, and benzothienyl.

A “heteroaralkyl” or “heteroarylalkyl” is heteroaryl group connected, as a substituent, via an alkylene group. Examples include but are not limited to 2-thienylmethyl, 3-thienylmethyl, furylmethyl, thienylethyl, pyrrolylalkyl, pyridylalkyl, isoxazollylalkyl, and imidazolylalkyl. In some cases, the alkylene group is a lower alkylene group (i.e., a C₁₋₄ alkylene group).

As used herein, “carbocyclyl” means a non-aromatic cyclic ring or ring system containing only carbon atoms in the ring system backbone. When the carbocyclyl is a ring system, two or more rings may be joined together in a fused, bridged or spiro-connected fashion. Carbocyclyls may have any degree of saturation provided that at least one ring in a ring system is not aromatic. Thus, carbocyclyls include cycloalkyls, cycloalkenyls, and cycloalkynyls. The carbocyclyl group may have 3 to 20 carbon atoms, although the present definition also covers the occurrence of the term “carbocyclyl” where no numerical range is designated. The carbocyclyl group may also be a medium size carbocyclyl having 3 to 10 carbon atoms. The carbocyclyl group could also be a carbocyclyl having 3 to 6 carbon atoms. The carbocyclyl group may be designated as “C₃₋₆ carbocyclyl” or similar designations. Examples of carbocyclyl rings include, but are not limited to, cyclopropyl, cyclobutyl, cyclopentyl, cyclohexyl, cyclohexenyl, 2,3-dihydro-indene, bicycle[2.2.2]octanyl, adamantyl, and spiro[4.4]nonanyl.

A “(carbocyclyl)alkyl” is a carbocyclyl group connected, as a substituent, via an alkylene group, such as “C₄₋₁₀ (carbocyclyl)alkyl” and the like, including but not limited to, cyclopropylmethyl, cyclobutylmethyl, cyclopropylethyl, cyclopropylbutyl, cyclobutylethyl, cyclopropylisopropyl, cyclopentylmethyl, cyclopentylethyl, cyclohexylmethyl, cyclohexylethyl, cycloheptylmethyl, and the like. In some cases, the alkylene group is a lower alkylene group.

As used herein, “cycloalkyl” means a fully saturated carbocyclyl ring or ring system. Examples include cyclopropyl, cyclobutyl, cyclopentyl, and cyclohexyl.

As used herein, “cycloalkenyl” means a carbocyclyl ring or ring system having at least one double bond, wherein no ring in the ring system is aromatic. An example is cyclohexenyl.

As used herein, “heterocyclyl” means a non-aromatic cyclic ring or ring system containing at least one heteroatom in the ring backbone. Heterocyclyls may be joined together in a fused, bridged or spiro-connected fashion. Heterocyclyls may have any degree of saturation provided that at least one ring in the ring system is not aromatic. The heteroatom(s) may be present in either a non-aromatic or aromatic ring in the ring system. The heterocyclyl group may have 3 to 20 ring members (i.e., the number of atoms making up the ring backbone, including carbon atoms and heteroatoms), although the present definition also covers the occurrence of the term “heterocyclyl” where no numerical range is designated. The heterocyclyl group may also be a medium size heterocyclyl having 3 to 10 ring members. The heterocyclyl group could also be a heterocyclyl having 3 to 6 ring members. The heterocyclyl group may be designated as “3-6 membered heterocyclyl” or similar designations.

In various embodiments, a heterocyclyl contains from 1 to 4 heteroatoms, from 1 to 3 heteroatoms, from 1 to 2 heteroatoms, or 1 heteroatom. For example, in various embodiments, a heterocyclyl contains 1 to 4 nitrogen atoms, 1 to 3 nitrogen atoms, 1 to 2 nitrogen atoms, 2 nitrogen atoms and 1 sulfur or oxygen atom, 1 nitrogen atom and 1 sulfur or oxygen atom, or 1 sulfur or oxygen atom. In preferred six membered monocyclic heterocyclyls, the heteroatom(s) are selected from one up to three of O, N or S, and in preferred five membered monocyclic heterocyclyls, the heteroatom(s) are selected from one or two heteroatoms selected from O, N, or S. Examples of heterocyclyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, isoindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline.

A “(heterocyclyl)alkyl” is a heterocyclyl group connected, as a substituent, via an alkylene group. Examples include, but are not limited to, imidazolinylmethyl and indolinylethyl.

As used herein, “acyl” refers to —C(═O)R, wherein R is hydrogen, C₁₋₆ alkyl, C₂₋₆ alkenyl, C₂₋₆ alkynyl, C₃₋₇ carbocyclyl, aryl, 5-10 membered heteroaryl, and 5-10 membered heterocyclyl, as defined herein. Non-limiting examples include formyl, acetyl, propanoyl, benzoyl, and acryl.

An “O-carboxy” group refers to a “—OC(═O)R” group in which R is selected from hydrogen, C₁₋₆ alkyl, C₂₋₆ alkenyl, C₂₋₆ alkynyl, C₃₋₇ carbocyclyl, aryl, 5-10 membered heteroaryl, and 5-10 membered heterocyclyl, as defined herein.

A “C-carboxy” group refers to a “—C(═O)OR” group in which R is selected from hydrogen, C₁₋₆ alkyl, C₂₋₆ alkenyl, C₂₋₆ alkynyl, C₃₋₇ carbocyclyl, aryl, 5-10 membered heteroaryl, and 5-10 membered heterocyclyl, as defined herein. A non-limiting example includes carboxyl (i.e., —C(═O)OH).

A “cyano” group refers to a “—CN” group.

A “cyanato” group refers to an “—OCN” group.

An “isocyanato” group refers to a “—NCO” group.

A “thiocyanato” group refers to a “—SCN” group.

An “isothiocyanato” group refers to an “—NCS” group.

A “sulfinyl” group refers to an “—S(═O)R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

A “sulfonyl” group refers to an “—SO₂R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “S-sulfonamido” group refers to a “—SO₂NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-sulfonamido” group refers to a “—N(R_{A})SO₂R_{B}” group in which R_{A} and R_{b} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “O-carbamyl” group refers to a “—OC(═O)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-carbamyl” group refers to an “—N(R_{A})OC(═O)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆_o aryl, 5-10 membered heteroaryl, and 5-10 membered heterocyclyl, as defined herein.

An “O-thiocarbamyl” group refers to a “—OC(═S)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆_o aryl, 5-10 membered heteroaryl, and 5-10 membered heterocyclyl, as defined herein.

An “N-thiocarbamyl” group refers to an “—N(R_{A})OC(═S)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

A “C-amido” group refers to a “—C(═O)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-amido” group refers to a “—N(R_{A})C(═O)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “amino” group refers to a “—NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “aminoalkyl” group refers to an amino group connected via an alkylene group.

An “alkoxyalkyl” group refers to an alkoxy group connected via an alkylene group, such as a “C₂₋₈ alkoxyalkyl” and the like.

As used herein, a “natural amino acid side chain” refers to the side-chain substituent of a naturally occurring amino acid. Naturally occurring amino acids have a substituent attached to the α-carbon. Naturally occurring amino acids include Arginine, Lysine, Aspartic acid, Glutamic acid, Glutamine, Asparagine, Histidine, Serine, Threonine, Tyrosine, Cysteine, Methionine, Tryptophan, Alanine, Isoleucine, Leucine, Phenylalanine, Valine, Proline, and Glycine.

As used herein, a “non-natural amino acid side chain” refers to the side-chain substituent of a non-naturally occurring amino acid. Non-natural amino acids include β-amino acids (β³ and β²), Homo-amino acids, Proline and Pyruvic acid derivatives, 3-substituted Alanine derivatives, Glycine derivatives, Ring-substituted Phenylalanine and Tyrosine Derivatives, Linear core amino acids and N-methyl amino acids. Exemplary non-natural amino acids are available from Sigma-Aldridge, listed under “unnatural amino acids & derivatives.” See also, Travis S. Young and Peter G. Schultz, “Beyond the Canonical 20 Amino Acids: Expanding the Genetic Lexicon,” J. Biol. Chem. 2010 285: 11039-11044, which is incorporated by reference in its entirety.

As used herein, a substituted group is derived from the unsubstituted parent group in which there has been an exchange of one or more hydrogen atoms for another atom or group. Unless otherwise indicated, when a group is deemed to be “substituted,” it is meant that the group is substituted with one or more substitutents independently selected from C₁-C₆ alkyl, C₁-C₆ alkenyl, C₁-C₆ alkynyl, C₁-C₆ heteroalkyl, C₃-C₇ carbocyclyl (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 5-10 membered heterocyclyl (optionally substituted with halo, C₁-C₆ alkyl, C₁-C₆ alkoxy, C₁-C₆ haloalkyl, and C₁-C₆ haloalkoxy), 5-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), aryl(C₁-C₆)alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkyl (optionally substituted with halo, C₁-C₆ alkyl, C₁-C₆ alkoxy, C₁-C₆ haloalkyl, and C₁-C₆ haloalkoxy), halo, cyano, hydroxy, C₁-C₆ alkoxy, C₁-C₆ alkoxy(C₁-C₆)alkyl (i.e., ether), aryloxy, sulfhydryl (mercapto), halo(C₁-C₆)alkyl (e.g., —CF₃), halo(C₁-C₆)alkoxy (e.g., —OCF₃), C₁-C₆ alkylthio, arylthio, amino, amino(C₁-C₆)alkyl, nitro, 0-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, and oxo (═O). Wherever a group is described as “optionally substituted” that group can be substituted with the above substituents.

In some embodiments, substituted group(s) is (are) substituted with one or more substituent(s) individually and independently selected from C₁-C₄ alkyl, amino, hydroxy, and halogen.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene” or “alkenylene.”

When two R groups are said to form a ring (e.g., a carbocyclyl, heterocyclyl, aryl, or heteroaryl ring) “together with the atom to which they are attached,” it is meant that the collective unit of the atom and the two R groups are the recited ring. The ring is not otherwise limited by the definition of each R group when taken individually. For example, when the following substructure is present:

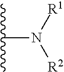

and R¹ and R² are defined as selected from the group consisting of hydrogen and alkyl, or R¹ and R² together with the nitrogen to which they are attached form a heterocyclyl, it is meant that R¹ and R² can be selected from hydrogen or alkyl, or alternatively, the substructure has structure:

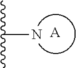

where ring A is a heterocyclyl ring containing the depicted nitrogen.

Similarly, when two “adjacent” R groups are said to form a ring “together with the atoms to which they are attached,” it is meant that the collective unit of the atoms, intervening bonds, and the two R groups are the recited ring. For example, when the following substructure is present:

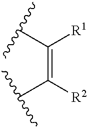

and R¹ and R² are defined as selected from the group consisting of hydrogen and alkyl, or R¹ and R² together with the atoms to which they are attached form an aryl or carbocyclyl, it is meant that R¹ and R² can be selected from hydrogen or alkyl, or alternatively, the substructure has structure:

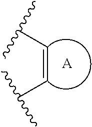

where A is an aryl ring or a carbocyclyl containing the depicted double bond.

Wherever a substituent is depicted as a di-radical (i.e., has two points of attachment to the rest of the molecule), it is to be understood that the substituent can be attached in any directional configuration unless otherwise indicated. Thus, for example, a substituent depicted as -AE- or

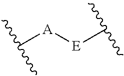

includes the substituent being oriented such that the A is attached at the leftmost attachment point of the molecule as well as the case in which A is attached at the rightmost attachment point of the molecule.

As used herein, “isosteres” of a chemical group are other chemical groups that exhibit the same or similar properties. For example, tetrazole is an isostere of carboxylic acid because it mimics the properties of carboxylic acid even though they both have very different molecular formulae. Tetrazole is one of many possible isosteric replacements for carboxylic acid. Other carboxylic acid isosteres contemplated include —SO₃H, —SO₂HNR, —PO₂(R)₂, —PO₃(R)₂, —CONHNHSO₂R, —COHNSO₂R, and —CONRCN, where R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein. In addition, carboxylic acid isosteres can include 5-7 membered carbocycles or heterocycles containing any combination of CH₂, O, S, or N in any chemically stable oxidation state, where any of the atoms of said ring structure are optionally substituted in one or more positions. The following structures are non-limiting examples of carbocyclic and heterocyclic isosteres contemplated. The atoms of said ring structure may be optionally substituted at one or more positions with R as defined above.

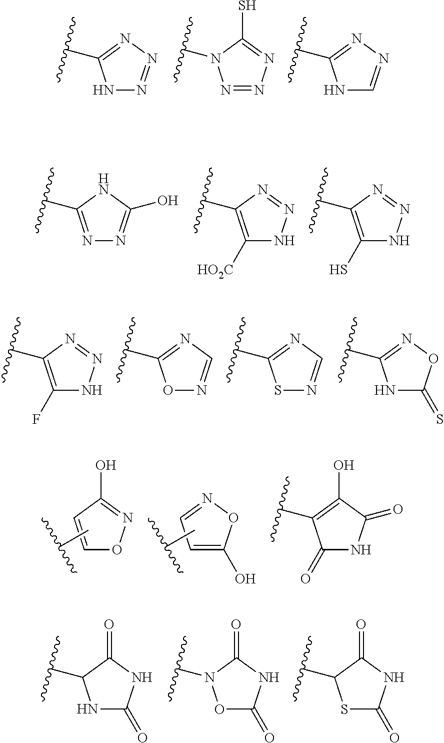

It is also contemplated that when chemical substituents are added to a carboxylic isostere, the compound retains the properties of a carboxylic isostere. It is contemplated that when a carboxylic isostere is optionally substituted with one or more moieties selected from R as defined above, then the substitution and substitution position is selected such that it does not eliminate the carboxylic acid isosteric properties of the compound. Similarly, it is also contemplated that the placement of one or more R substituents upon a carbocyclic or heterocyclic carboxylic acid isostere is not a substitution at one or more atom(s) that maintain(s) or is/are integral to the carboxylic acid isosteric properties of the compound, if such substituent(s) would destroy the carboxylic acid isosteric properties of the compound.

Other carboxylic acid isosteres not specifically exemplified in this specification are also contemplated.

The term “agent” or “test agent” includes any substance, molecule, element, compound, entity, or a combination thereof. It includes, but is not limited to, e.g., protein, polypeptide, peptide or mimetic, small organic molecule, polysaccharide, polynucleotide, and the like. It can be a natural product, a synthetic compound, or a chemical compound, or a combination of two or more substances. Unless otherwise specified, the terms “agent”, “substance”, and “compound” are used interchangeably herein.

The term “analog” is used herein to refer to a molecule that structurally resembles a reference molecule but which has been modified in a targeted and controlled manner, by replacing a specific substituent of the reference molecule with an alternate substituent. Compared to the reference molecule, an analog would be expected, by one skilled in the art, to exhibit the same, similar, or improved utility. Synthesis and screening of analogs, to identify variants of known compounds having improved characteristics (such as higher binding affinity for a target molecule) is an approach that is well known in pharmaceutical chemistry.

### Compounds

In some embodiments, the calpain inhibitor may be selected from a compound having the structure of the Formula I:

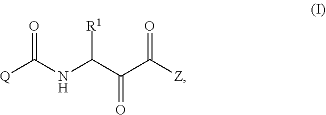

or a pharmaceutically acceptable salt or prodrug thereof, wherein:

R¹ is —C₁₋₆alkyl or —(CH₂)ₙ—C₆₋₁₀aryl optionally substituted with one, two, or three substituents independently selected from the group consisting of: -halo, —C₁₋₆ alkyl, —C₁₋₆haloalkyl, —C₁₋₆alkoxy, and —C₁₋₆haloalkoxy;

Z is —NR²R³ or —OR⁴;

R² is -hydrogen or —C₁₋₆ alkyl;

R³ is -hydrogen, —C₁₋₆ alkyl, —C₃₋₁₀ cycloalkyl, or —OR⁴;

R⁴ is -hydrogen or —C₁₋₆ alkyl;

Q is -5-10-membered heteroaryl optionally substituted with one, two, or three substituents independently selected from the group consisting of: -halo; —C₁₋₆ alkyl; —C₁₋₆ haloalkyl; —C₁₋₆ alkoxy; —C₁₋₆ haloalkoxy; and —C₆₋₁₀ aryl optionally substituted with one, two, or three substituents independently selected from the group consisting of: -halo, —C₁₋₆ alkyl, —C₁₋₆ haloalkyl, —C₁₋₆alkoxy, and —C₁₋₆haloalkoxy; or

Q is —C₆₋₁₀aryl optionally substituted with one, two, or three substituents independently selected from the group consisting of: -halo; —C₁₋₆ alkyl; —C₁₋₆ haloalkyl; —C₁₋₆alkoxy; —C₁₋₆haloalkoxy; and -5-10-membered heteroaryl optionally substituted with one, two, or three substituents independently selected from the group consisting of: -halo, —C₁₋₆ alkyl, —C₁₋₆ haloalkyl, —C₁₋₆alkoxy, and —C₁₋₆haloalkoxy; and

n is 1 or 2.

In some embodiments, the compound of Formula (1) has the Formula (1-a)

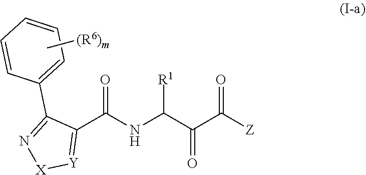

wherein:

X is S or NR⁵;

Y is CH or N;

R⁵ is -hydrogen or —C₁₋₆ alkyl;

each R⁶ is independently selected from the group consisting of: -halo, —C₁₋₆ alkyl, —C₁₋₆ haloalkyl, —C₁₋₆ alkoxy, and —C₁₋₆ haloalkoxy; and

m is 0, 1, 2, or 3.

In some embodiments, X may be S. In other embodiments, X may be NR⁵. In some specific embodiments, R⁵ may be hydrogen or —CH₃.

In some embodiments, Y may be CH. In other embodiments, Y may be N.

In some embodiments, X may be S and Y may be N. In other embodiments, X may be NR⁵ and Y may be CH.

In some embodiments, R⁶ may independently be —F, —Cl, —CH₃, —CF₃, —OCH₃, or —OCF₃.

In some embodiments, wherein m may be 0, 1, 2, or 3.

In some embodiments, the compound of Formula (1) has the Formula (1-b)

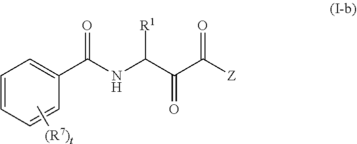

wherein:

each R⁷ is independently selected from the group consisting of: -halo, —C₁₋₆ alkyl, —C₁₋₆ haloalkyl, —C₁₋₆ alkoxy, and —C₁₋₆ haloalkoxy; and

t is 0, 1, 2, or 3.

In some embodiments, each R⁷ may independently be —F, —Cl, —CH₃, —CF₃, —OCH₃, or —OCF₃.

In some embodiments, t may be 0, 1, 2, or 3.

In some embodiments described herein, Z may be —NR²R³. In some embodiments, R² may be -hydrogen. In some embodiments, R³ may be -hydrogen, —CH₃, or -cyclopropyl. In other embodiments, R³ may be —OH or —OCH₃.

In some embodiments, Z may be —OR⁴. In some embodiments, R⁴ may be -hydrogen or —CH₃.

In some embodiments, the compound may be selected from the group consisting of:

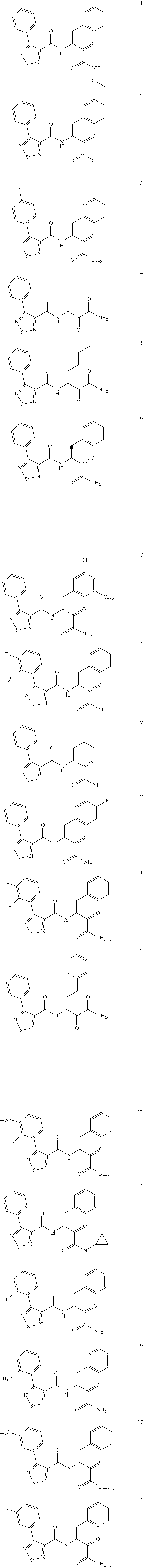

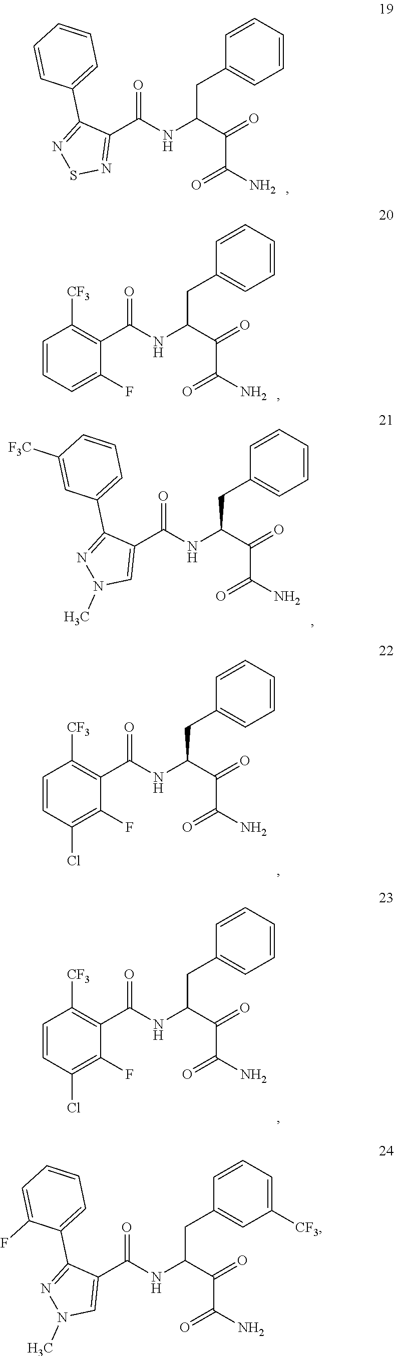

and pharmaceutically acceptable salts thereof.

### Second Pharmaceutical Agents

The compounds presented herein may be administered in combination with one or more second pharmaceutical agents. In some embodiments, the compounds described above may be administered in combination with one second pharmaceutical agent. In some embodiments, the compounds described above may be administered in combination with two second pharmaceutical agents. In some embodiments, the compounds described above may be administered in combination with three or more second pharmaceutical agents.

In some embodiments, the compounds presented herein may be administered simultaneously with one or more second pharmaceutical agents. In other embodiments, the compounds of the present disclosure may be administered sequentially with one or more second pharmaceutical agents.

In some embodiments, the second pharmaceutical agent may be: a vesicular monoamine transporter 2 inhibitor including but not limited to tetrabenazine and deutetrabenazine; an antidepressant including, but not limited to citalopran, escitalipram, fluoxetine, and sertraline; an antipsychotic agent including but not limited to quetiapine, risperidone, haloperidol, and chlorpromazine: or a mood-stabilizing agent including but not limited to valproate, carbanazepine, and lamotrigine. Additional second pharmaceutical agents include but are not limited to: levodopa, baclofen, and botulinum toxin.

### Methods of Treatment

In some embodiments, the compounds disclosed herein are calpain inhibitors. In some embodiments, the compounds can effectively act as CAPN1, CAPN2, and/or CAPN9 inhibitors. Some embodiments provide pharmaceutical compositions comprising one or more compounds disclosed herein and a pharmaceutically acceptable excipient.

In some embodiments, the compounds and compositions comprising the compounds described herein can be used to treat a host of neurological conditions arising from expanded polyglutamine tracts in some proteins. Example conditions include, but are not limited to, Huntington's disease, Machado-Joseph disease, dentatorubral-pallidoluysian atrophy, spinal and bulbar muscular atrophy, spinocerebellar ataxia type 1 (SCA1), spinocerebellar ataxia type 2 (SCA2), spinocerebellar ataxia type 6 (SCA6), spinocerebellar ataxia type 7 (SCA7), spinocerebellar ataxia type 17 (SCA17).

In some embodiments, the compounds and pharmaceutical compositions comprising compounds disclosed herein may be used to treat Huntington's disease. Without being limited by a particular theory, calpain-mediated cleavage of huntingtin protein (Htt) may contribute to neurodegeneration and progression of Huntington's disease. Calpain-resistant Htt mutant cells exhibit reduced Htt aggregation and cellular toxicity vs. wild-type Htt. Gafni et al, J. Biol. Chem. 2004, 279, 20211-20220. Calpain activity and Htt proteolysis are increased in the striatum and cortex in an HD knock-in murine model. Overexpression of calpastatin, an endogenous calpain inhibitor, ameliorates disease pathogenesis and symptoms in mice, while ablation of calpastatin exacerbates Htt aggregation in cells and mice. Menzies et al., Cell Death Diff. 2015, 22, 433-444; Weber et al, Neuropharmacol. 2008, 133(1), 94-106. Calpain activation is increased in human Huntington's disease patients as compared to controls. Moreover, a major Htt fragment in HD tissue appears to be derived from calpain-cleavage-Gafni et al, J. Neurosci. 2002, 22(12), 4842-4849.

In some embodiments, the compounds and pharmaceutical compositions comprising compounds disclosed herein may be used to treat Machado-Joseph disease (MJD). Without being limited by a particular theory, calpains cleave ataxin-3 in expanded polyglutamine repeats and calpain inhibition abrogates fragmentation and formation of neuronal inclusions in MJD cells. Haacke et al., J. Biol. Chem. 2007, 282, 18851-18856; Hubener, 2013, Hum Mol Genetics; Koch, 2011, Nature. Ablation of calpastatin led to increased mutant ataxin-3 fragments, nuclear inclusions, and neurodegeneration in MJD mice. Hubener, 2013, Hum Mol Genetics. Calpastatin overexpression reduced the size and number of mutant ataxin-3 inclusions and neurodegeneration in MJD mice (Simoes, 2012, Brain) and administration of a calpain inhibitor reduced mutant ataxin-3 aggregation and cellular degeneration and prevented motor behavioral deficits (Simoes, 2014, Hum Mol Genetics). Calpain also cleaves ataxin-3 in MJD patient-derived cells and post mortem brain tissue. Weber, 2017, Brain.

In some embodiments, the compounds and pharmaceutical compositions comprising compounds disclosed herein may be used to treat other neurological diseases or disorders associated with protein aggregation. Example conditions include, but are not limited to, amyotrophic lateral sclerosis, Alzheimer's disease, Parkinson's disease, frontal temporal dementia, and prion disease.

### Administration and Pharmaceutical Compositions

The compounds are administered at a therapeutically effective dosage. While human dosage levels have yet to be optimized for the compounds described herein, generally, a daily dose may be from about 0.25 mg/kg to about 120 mg/kg or more of body weight, from about 0.5 mg/kg or less to about 70 mg/kg, from about 1.0 mg/kg to about 50 mg/kg of body weight, or from about 1.5 mg/kg to about 10 mg/kg of body weight. Thus, for administration to a 70 kg person, the dosage range would be from about 17 mg per day to about 8000 mg per day, from about 35 mg per day or less to about 7000 mg per day or more, from about 70 mg per day to about 6000 mg per day, from about 100 mg per day to about 5000 mg per day, or from about 200 mg to about 3000 mg per day. The amount of active compound administered will, of course, be dependent on the subject and disease state being treated, the severity of the affliction, the manner and schedule of administration and the judgment of the prescribing physician.

Administration of the compounds disclosed herein or the pharmaceutically acceptable salts thereof can be via any of the accepted modes of administration for agents that serve similar utilities including, but not limited to, orally, subcutaneously, intravenously, intranasally, topically, transdermally, intraperitoneally, intramuscularly, intrapulmonarilly, vaginally, rectally, or intraocularly.

The compounds useful as described above can be formulated into pharmaceutical compositions for use in treatment of these conditions. Standard pharmaceutical formulation techniques are used, such as those disclosed in Remington's The Science and Practice of Pharmacy, 21st Ed., Lippincott Williams & Wilkins (2005), incorporated by reference in its entirety. Accordingly, some embodiments include pharmaceutical compositions comprising: (a) a safe and therapeutically effective amount of a compound described herein (including enantiomers, diastereoisomers, tautomers, polymorphs, and solvates thereof), or pharmaceutically acceptable salts thereof; and (b) a pharmaceutically acceptable carrier, diluent, excipient or combination thereof.

In addition to the selected compound useful as described above, come embodiments include compositions containing a pharmaceutically-acceptable carrier. The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

Some examples of substances, which can serve as pharmaceutically-acceptable carriers or components thereof, are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the TWEENS; wetting agents, such sodium lauryl sulfate; coloring agents; flavoring agents; tableting agents, stabilizers; antioxidants; preservatives; pyrogen-free water; isotonic saline; and phosphate buffer solutions.

The choice of a pharmaceutically-acceptable carrier to be used in conjunction with the subject compound is basically determined by the way the compound is to be administered.

The compositions described herein are preferably provided in unit dosage form. As used herein, a “unit dosage form” is a composition containing an amount of a compound that is suitable for administration to an animal, preferably mammal subject, in a single dose, according to good medical practice. The preparation of a single or unit dosage form however, does not imply that the dosage form is administered once per day or once per course of therapy. Such dosage forms are contemplated to be administered once, twice, thrice or more per day and may be administered as infusion over a period of time (e.g., from about 30 minutes to about 2-6 hours), or administered as a continuous infusion, and may be given more than once during a course of therapy, though a single administration is not specifically excluded. The skilled artisan will recognize that the formulation does not specifically contemplate the entire course of therapy and such decisions are left for those skilled in the art of treatment rather than formulation.

The compositions useful as described above may be in any of a variety of suitable forms for a variety of routes for administration, for example, for oral, nasal, rectal, topical (including transdermal), ocular, intracerebral, intracranial, intrathecal, intra-arterial, intravenous, intramuscular, or other parental routes of administration. The skilled artisan will appreciate that oral and nasal compositions comprise compositions that are administered by inhalation, and made using available methodologies. Depending upon the particular route of administration desired, a variety of pharmaceutically-acceptable carriers well-known in the art may be used. Pharmaceutically-acceptable carriers include, for example, solid or liquid fillers, diluents, hydrotropies, surface-active agents, and encapsulating substances. Optional pharmaceutically-active materials may be included, which do not substantially interfere with the inhibitory activity of the compound. The amount of carrier employed in conjunction with the compound is sufficient to provide a practical quantity of material for administration per unit dose of the compound. Techniques and compositions for making dosage forms useful in the methods described herein are described in the following references, all incorporated by reference herein: Modern Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004).

Various oral dosage forms can be used, including such solid forms as tablets, capsules, granules and bulk powders. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

The pharmaceutically-acceptable carrier suitable for the preparation of unit dosage forms for peroral administration is well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical, and can be readily made by a person skilled in the art.

Peroral compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, AVICEL RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Such compositions may also be coated by conventional methods, typically with pH or time-dependent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit coatings, waxes and shellac.

Compositions described herein may optionally include other drug actives.

Other compositions useful for attaining systemic delivery of the subject compounds include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

A liquid composition, which is formulated for topical ophthalmic use, is formulated such that it can be administered topically to the eye. The comfort should be maximized as much as possible, although sometimes formulation considerations (e.g. drug stability) may necessitate less than optimal comfort. In the case that comfort cannot be maximized, the liquid should be formulated such that the liquid is tolerable to the patient for topical ophthalmic use. Additionally, an ophthalmically acceptable liquid should either be packaged for single use, or contain a preservative to prevent contamination over multiple uses.

For ophthalmic application, solutions or medicaments are often prepared using a physiological saline solution as a major vehicle. Ophthalmic solutions should preferably be maintained at a comfortable pH with an appropriate buffer system. The formulations may also contain conventional, pharmaceutically acceptable preservatives, stabilizers and surfactants.

Preservatives that may be used in the pharmaceutical compositions disclosed herein include, but are not limited to, benzalkonium chloride, PHMB, chlorobutanol, thimerosal, phenylmercuric, acetate and phenylmercuric nitrate. A useful surfactant is, for example, Tween 80. Likewise, various useful vehicles may be used in the ophthalmic preparations disclosed herein. These vehicles include, but are not limited to, polyvinyl alcohol, povidone, hydroxypropyl methyl cellulose, poloxamers, carboxymethyl cellulose, hydroxyethyl cellulose and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. For many compositions, the pH will be between 4 and 9. Accordingly, buffers include acetate buffers, citrate buffers, phosphate buffers and borate buffers. Acids or bases may be used to adjust the pH of these formulations as needed.

In a similar vein, an ophthalmically acceptable antioxidant includes, but is not limited to, sodium metabisulfite, sodium thiosulfate, acetylcysteine, butylated hydroxyanisole and butylated hydroxytoluene.

Other excipient components, which may be included in the ophthalmic preparations, are chelating agents. A useful chelating agent is edetate disodium, although other chelating agents may also be used in place or in conjunction with it.

For topical use, creams, ointments, gels, solutions or suspensions, etc., containing the compound disclosed herein are employed. Topical formulations may generally be comprised of a pharmaceutical carrier, co-solvent, emulsifier, penetration enhancer, preservative system, and emollient.

For intravenous administration, the compounds and compositions described herein may be dissolved or dispersed in a pharmaceutically acceptable diluent, such as a saline or dextrose solution. Suitable excipients may be included to achieve the desired pH, including but not limited to NaOH, sodium carbonate, sodium acetate, HCl, and citric acid. In various embodiments, the pH of the final composition ranges from 2 to 8, or preferably from 4 to 7. Antioxidant excipients may include sodium bisulfite, acetone sodium bisulfite, sodium formaldehyde, sulfoxylate, thiourea, and EDTA. Other non-limiting examples of suitable excipients found in the final intravenous composition may include sodium or potassium phosphates, citric acid, tartaric acid, gelatin, and carbohydrates such as dextrose, mannitol, and dextran. Further acceptable excipients are described in Powell, et al., Compendium of Excipients for Parenteral Formulations, PDA J Pharm Sci and Tech 1998, 52 238-311 and Nema et al., Excipients and Their Role in Approved Injectable Products: Current Usage and Future Directions, PDA J Pharm Sci and Tech 2011, 65 287-332, both of which are incorporated herein by reference in their entirety. Antimicrobial agents may also be included to achieve a bacteriostatic or fungistatic solution, including but not limited to phenylmercuric nitrate, thimerosal, benzethonium chloride, benzalkonium chloride, phenol, cresol, and chlorobutanol.

The compositions for intravenous administration may be provided to caregivers in the form of one more solids that are reconstituted with a suitable diluent such as sterile water, saline or dextrose in water shortly prior to administration. In other embodiments, the compositions are provided in solution ready to administer parenterally. In still other embodiments, the compositions are provided in a solution that is further diluted prior to administration. In embodiments that include administering a combination of a compound described herein and another agent, the combination may be provided to caregivers as a mixture, or the caregivers may mix the two agents prior to administration, or the two agents may be administered separately.

The actual dose of the active compounds described herein depends on the specific compound, and on the condition to be treated; the selection of the appropriate dose is well within the knowledge of the skilled artisan.

The compounds and compositions described herein, if desired, may be presented in a pack or dispenser device containing one or more unit dosage forms containing the active ingredient. Such a pack or device may, for example, comprise metal or plastic foil, such as a blister pack, or glass, and rubber stoppers such as in vials. The pack or dispenser device may be accompanied by instructions for administration. Compounds and compositions described herein are formulated in a compatible pharmaceutical carrier may also be prepared, placed in an appropriate container, and labeled for treatment of an indicated condition.

The amount of the compound in a formulation can vary within the full range employed by those skilled in the art. Typically, the formulation will contain, on a weight percent (wt %) basis, from about 0.01 to about 99.99 wt % of a compound of the present technology based on the total formulation, with the balance being one or more suitable pharmaceutical excipients. Preferably, the compound is present at a level of about 1 to about 80 wt %. Representative pharmaceutical formulations are described below.

### FORMULATION EXAMPLES

The following are representative pharmaceutical formulations containing a compound of Formula I.

### Formulation Example 1—Tablet Formulation

The following ingredients are mixed intimately and pressed into single scored tablets.

| | Quantity per |
|---|---|
| Ingredient | tablet, mg |
| Compounds disclosed herein | 400 |
| cornstarch | 50 |
| croscarmellose sodium | 25 |
| lactose | 120 |
| magnesium stearate | 5 |

### Formulation Example 2—Capsule Formulation

The following ingredients are mixed intimately and loaded into a hard-shell gelatin capsule.

| | Quantity per |
|---|---|
| Ingredient | capsule, mg |
| Compounds disclosed herein | 200 |
| lactose, spray-dried | 148 |
| magnesium stearate | 2 |

### Formulation Example 3—Suspension Formulation

The following ingredients are mixed to form a suspension for oral administration.

| Ingredient | Amount | |
|---|---|---|
| Compounds disclosed herein | 1.0 | g |
| fumaric acid | 0.5 | g |
| sodium chloride | 2.0 | g |
| methyl paraben | 0.15 | g |
| propyl paraben | 0.05 | g |
| granulated sugar | 25.0 | g |
| sorbitol (70% solution) | 13.00 | g |
| Veegum K (Vanderbilt Co.) | 1.0 | g |
| flavoring | 0.035 | mL |
| colorings | 0.5 | mg |

| | |
|---|---|
| distilled water | q.s. to 100 mL |

### Formulation Example 4—Injectable Formulation

The following ingredients are mixed to form an injectable formulation.

| Ingredient | Amount |
|---|---|
| Compounds disclosed herein | 0.2 mg-20 mg |
| sodium acetate buffer solution, 0.4M | 2.0 mL |
| HCl (1N) or NaOH (1N) | q.s. to suitable pH |
| water (distilled, sterile) | q.s. to 20 mL |

### Formulation Example 5—Suppository Formulation

A suppository of total weight 2.5 g is prepared by mixing the compound of the present technology with Witepsol® H-15 (triglycerides of saturated vegetable fatty acid; Riches-Nelson, Inc., New York), and has the following composition:

| Ingredient | Amount |
|---|---|
| Compounds disclosed herein | 500 mg |
| Witepsol ® H-15 | balance |

The following examples are included for illustrative purposes. The examples should not, of course, be construed as specifically limiting the scope of the disclosure. Variations of these examples within the scope of the claims are within the purview of one skilled in the art and are considered to fall within the scope of the disclosure as described, and claimed herein. The reader will recognize that the skilled artisan, armed with the present disclosure, and skill in the art is able to prepare and use the subject matter described herein without exhaustive examples. The following examples will further describe the present disclosure, and are used for the purposes of illustration only, and should not be considered as limiting.

### EXAMPLES

### Example 1-Calpain Inhibition

Calpain 1, 2, and 9 activity and inhibition thereof was assessed by means of a continuous fluorescence assay. The SensoLyte 520 Calpain substrate (Anaspec Inc) was optimized for detecting calpain activity. This substrate contains an internally quenched 5-FAM/QXLTM 520 FRET pair. Calpains 1, 2, and 9 cleave the FRET substrate into two separate fragments resulting in an increase of 5-FAM fluorescence that is proportional to calpain activity

Assays were typically setup in black 384-well plates using automated liquid handling as follows. Calpain assay base buffer typically contains 50 mM Tris, pH 7.5, 100 mM NaCl and 1 mM DTT. Inhibitors were serially diluted in DMSO and used to setup 2× mixtures with calpains in the aforementioned buffer. After incubation at ambient temperature (25° C.), the reaction was initiated by adding a 2× mix of the fluorescent peptide substrate and CaCl₂) (required for in-situ calpain activation) in the same buffer. Reaction progress curve data were typically collected for 10 min using excitation/emission wavelengths of 490 nm/520 nm on SpectraMax i3x or the FLIPR-Tetra plate readers (Molecular Devices Inc). Reaction rates were calculated from progress curve slopes typically over 1-5 min. Dose response curves (rate vs. log inhibitor concentration) were typically fit to a 4-parameter logistic function to extract IC₅₀ values.

Calpain activity in SH-SY5Y cells and inhibition thereof were assessed by means of a homogeneous, fluorescence assay that uses the cell-permeable and pro-fluorescent calpain substrate Suc-LLVY-AMC (Sigma-Aldrich Inc). Upon intracellular calpain cleavage of Suc-LLVY-AMC, fluorescent amino-methyl-coumarin (AMC) is released into the media resulting in a continuous increase in fluorescence signal that is proportional to intra-cellular calpain activity.

Assays were typically setup by seeding SH-SY5Y cells in black 384-well plates at 40 k/per well in RPMI-1640 containing 1% serum followed by 37° C. overnight incubation. The next morning, cells were pre-incubated for 30 min with serially diluted compounds followed by addition of 100 uM of Suc-LLVY-AMC substrate. The continuous increase in AMC fluorescence is monitored using a FLIPR Tetra plate reader (Molecular Devices Inc) and slopes measured to report calpain activity. Dose response curves (slopes vs. log inhibitor concentration) were typically fit to a 4-parameter logistic function to extract IC50 values.

Calpain activity in SH-SY5Y cells and inhibition thereof were also assessed by a western blot based assay that measures a calpain-specific breakdown product of the alpha chain of non-erythrocytic spectrin (SBDP-150). Addition of the calcium ionophore A23187 was used to induce calpain activity and SBDP-150 formation.

These assays were set up by adding SH-SY5Y cells in 96-well plates at 250 k/per well in serum-free MEM and F12 media (1:1 mixture) with 3 mM calcium chloride. The cells were then pre-incubated for 20 minutes with serially diluted compounds followed by addition of 5 CaM A23187 and further incubation for 30 minutes. After plate centrifugation, media supernatant was removed. Cells pellets were lysed in MPER buffer containing 5 mM EDTA and Protease-Phosphatase inhibitor cocktail mixture (Thermofisher Inc) and stored at −80° C. until analysis. Lysate samples were thawed and centrifuged, and supernatants were used for spectrin breakdown product (SBDP) quantitation using the AA6 antibody (Enzo Inc.) on the Jess platform (Protein Simple Inc.). GAPDH and HSP60 were measured as internal reference proteins. Normalized SBDP levels vs. log inhibitor concentration were plotted to get dose response curves that are typically fit to a 4-parameter logistic function to extract IC₅₀ values.

TABLE 1
Calpain Inhibition Assay Data

| | Human | Human | Human | SH- | SH-SY5Y + |
|---|---|---|---|---|---|
| | Calpain | Calpain | Calpain | SY5Y | AMC |
| Com- | 1/NS1 | 9/NS1 | 2/NS1 | Spectrin | (basal) |
| pound | IC₅₀ | IC₅₀ | IC₅₀ (nM) | IC₅₀ | IC₅₀ |
| 1 | C | B | C | ND | E |
| 2 | B | C | B | E | D |
| 3 | B | B | A | E | E |
| 4 | C | C | C | F | F |
| 5 | A | B | A | F | F |
| 6 | A | A | A | E | E |
| 7 | B | B | B | D | F |
| 8 | C | C | C | E | E |
| 9 | A | A | A | E | F |
| 10 | B | B | A | D | E |
| 11 | B | B | B | E | E |
| 12 | B | B | A | F | F |
| 13 | B | B | A | E | D |
| 14 | C | C | C | E | ND |
| 15 | B | B | B | E | D |
| 16 | C | C | C | F | E |
| 17 | A | B | A | E | F |
| 18 | B | C | B | E | F |
| 19 | B | B | B | E | E |
| 20 | C | B | C | ND | F |
| 21 | C | A | C | F | F |
| 22 | A | A | A | E | E |
| 23 | A | B | A | E | E |
| 24 | C | C | C | F | E |

### Example 2—Neurological Data

The compounds disclosed herein are orally administered to the mouse models described below (30 mg/kg in 1% Tween 80 saline in a volume equal to 5 mL/kg), with a 20 G gavage needle, every day since 2 days before stereotaxic injection until sacrifice. The analyses described below are conducted on the mice and compared to controls not receiving the test compounds.

### Cultures of Cerebellar Gzranule Neurons

Primary cultures of rat cerebellar granule neurons are prepared from P7 post-natal Wistar rat pups. Cerebella are dissected and dissociated with trypsin (0.01%, 15 min, and 37° C., Sigma, T0303) and DNase (0.045 mg/ml, Sigma, D5025) in Ca²⁺- and Mg²⁺-free Krebs buffer (120 mM NaCl, 5 mM KCl, 1.2 mM KH₂PO₄, 13 mM glucose, 15 mM 4-(2-hydroxyethyl)piperazine-1-ethanesulfonic acid (HEPES), 0.3% BSA, pH 7.4). Cerebella are then washed with Krebs buffer containing trypsin inhibitor (0.3 mg/ml, Sigma, T9128) to stop trypsin activity. The cells are dissociated in this solution, centrifuged and are then resuspended in Basal Medium Eagle supplemented with 25 mM KCl, 30 mM glucose, 26 mM NaHCO₃, 1% penicillin-streptomycin (100 U/ml, 100 mg/ml) and 10% fetal bovine. Cells are plated on 6 or 12-well plates (1×106 or 5×105 cells/well) coated with poly-D-lysine. Cultures are maintained for 3 weeks in a humid incubator (5% CO₂/95% air at 37° C.).

### Animals

Four-week-old C₅₇BL/6J mice (Charles River) are were used. The animals are housed in a temperature-controlled room maintained on a 12 h light/12 h dark cycle. Food and water are provided ad libitum. The experiments are carried out in accordance with the European Union Directive 2010/63/EU covering the protection of animals used for scientific purposes.

### Viral Vectors Production

Lentiviral vectors encoding human wild-type ataxin-3 (ATX-3 27Q) or mutant ataxin-3 (ATX-3 72Q) are produced in 293T cells with a four-plasmid system, as previously described in de Almeida et al, Neurobiol. Dis., 2001, 8, 433-446. The lentiviral particles are resuspended in 1% bovine serum albumin (BSA) in phosphate-buffered saline (PBS). The viral particle content of batches is determined by assessing HIV-1 p24 antigen levels (RETROtek, Gentaur, Paris, France). Viral stocks is stored at −80° C. until use.

### Lentiviral Infection of Cerebellar Granule Neurons

The cell cultures are infected with lentiviral vectors at ratio of 10 ng of p24 antigen/105 cells 1 day after plating (1 DIV) (See Zala et al., Neurobiol. Dis. 2005, 20, 785-798). At 2 DIV, medium is replaced with freshly prepared culture medium and a compound of the instant disclosure is added in two different concentrations (50 and 100 nM in DMSO). DMSO is used as a control. Medium plus inhibitor or DMSO is replaced every 3 days.

### In Vivo Injection in the Striatum and Cerebellum

Concentrated viral stocks are thawed on ice. Lentiviral vectors encoding human wild-type (ATX-3 27Q) or mutant ataxin-3 (ATX-3 72Q) are stereotaxically injected into the striatum in the following coordinates: antero-posterior: +0.6 mm; medial-lateral: +1.8 mm; dorsoventral: 23.3 mm; and into the cerebellum in the following coordinates: anteroposterior: 22.4 mm; medial-lateral: 0 mm; dorsoventral: 22.9 mm. Animals are anesthetized by administration of avertin (200 mg/g, intraperitoneally).

For western-blot procedure and RNA extraction, wild-type mice receive a single 2 mL injection of 0.3 mg of p24/ml lentivirus in each side: left hemisphere (ATX-3 27Q) and right hemisphere (ATX-3 72Q). For immunohistochemical procedure, wild-type mice receive a single 1 ml injection of 0.4 mg of p24/mL lentivirus in each side: left hemisphere (ATX-3 27Q) and right hemisphere (ATX-3 72Q). Mice are kept in their home cages for 4 or 8 weeks, before being sacrifice for western-blot analysis and RNA extraction or immunohistochemical analysis, respectively.

For behavioural analysis and cerebellar morphologic assessment, wild-type mice receive a single 4 mL injection of 0.25 mg of p24/ml lentivirus encoding ATX-3 72Q. Noninjected mice (Ø) of the same age are used as a control.

### Behavioral Assessments

Mice are subjected to locomotor tests starting at 4 weeks of age. Animals are habituated for 1 h to a quiet room with controlled temperature and ventilation, dimmed lighting, and handled prior to behavioral testing to overcome the animals' natural fear and anxiety responses, which could have a major effect on performance. All devices are wiped clean with a damp cloth of a 10% ethanol solution and dried before evaluating the next mouse.

Beam balance/walking: Motor coordination and balance of mice are assessed by measuring the ability of the mice to traverse a graded series of narrow beams to reach an enclosed safety platform (Carter et al., J. Neurosci., 1999, 19, 3248-3257). The beams consist of long strips of wood (1 m) with an 18 or 9 mm square wide and a 9 or 6 mm round diameter cross-sections. The beams are placed horizontally, 25 cm above the bench surface, with one end mounted on a narrow support and the other end attached to an enclosed box (20 cm square) into which the mouse could escape. A 60 W desk lamp is positioned above near the start of the beam to create an aversive stimulus (bright light) to induce mice to cross it. Mice perform two consecutive trials on each beam, progressing from the widest to the narrowest beam and the mean taken to analysis. The mean latency time each animal spent to cross all the beams is considered.

Grip strength: The mouse limb strength is measured as an indicator of neuromuscular function. The setup consists of a 300 g metal grid, which is on a scale. The animal is hung with its forepaws on the central position of the grid. Its strength is determined as the weight pushed (g) from the scale. The grip test is performed three times and the mean is taken to analysis. Mice body weight is used as a normalization factor.

Footprint test: Gait analysis is assessed by the footprint test. To obtain footprints, the hindfeet and forefeet of the mice are coated with black and white non-toxic paints, respectively. Mice are allowed to walk on a greenish paper along a 100×10×15 cm runway. Stride length is measured as the average distance of forward movement between each stride. A sequence of six consecutive steps for both hind and forefeet is chosen for evaluation. The mean of the 12 strides for each animal is considered.

### Immunohistochemical Procedure

After an overdose of avertin (2.5×200 mg/g, i.p.), transcardial perfusion of the mice is performed with a phosphate solution followed by fixation with 4% paraformaldehyde (PFA). The brains are removed and post-fixed in 4% PFA for 24 h and cryoprotected by incubation in 25% sucrose/phosphate buffer for 48 h. The brains are frozen, 25 mm coronal striatal sections and 35 mm midsagittal cerebellar sections are cut using a cryostat (LEICA CM3050 S) at −80° C. Slices throughout the entire brain regions are collected in anatomical series and stored in 48-well trays as free-floating sections in PBS supplemented with 0.05 mM sodium azide. The trays are stored at 4° C. until immunohistochemical processing.

Sections from injected mice are processed with the following primary antibodies: a mouse monoclonal anti-ataxin-3 antibody (1H9, 1:5000; Chemicon, Temecula, Calif.), recognizing the human ataxin-3 fragment from amino acids F112-L249; a rabbit polyclonal anti-ubiquitin antibody (Dako, 1:1000; Cambridgeshire, UK); and a rabbit anti-DARPP-32 antibody (1:1000; Chemicon, Temecula, Calif.), followed by incubation with the respective biotinylated secondary antibodies (1:200; Vector Laboratories). Bound antibodies are visualized using the Vectastain ABC kit, with 3,3′-diaminobenzidine tetrahydrochloride (DAB metal concentrate; Pierce) as substrate.

Double stainings for Ataxin-3 (1H9, 1:3000; Chemicon, Temecula, Calif.), nuclear marker [4′,6-diamidino-2-phenylindole (DAPI), blue] and ubiquitin (Dako, 1:1000; Cambridgeshire, UK), cleaved caspase-3 (Asp175, 1:2000; Cell Signaling) or calbindin (Ab1778, 1:500; Chemicon, Temecula, Calif.) are performed. Free-floating sections from injected mice are at room temperature (RT) for 2 h in PBS/0.1% Triton X-100 containing 10% NGS (Gibco), and then overnight at 4° C. in blocking solution with the primary antibodies. Sections are washed three times and incubated for 2 h at RT with the corresponding secondary antibodies coupled to fluorophores (1:200; Molecular Probes, Oregon, USA) diluted in the respective blocking solution. The sections are washed three times and then mounted in Fluorsave Reagent@ (Calbiochem, Germany) on microscope slides.

Staining is visualized using Zeiss Axioskop 2 plus, Zeiss Axiovert 200 and Zeiss LSM 510 Meta imaging microscopes (Carl Zeiss Microimaging, Germany), equipped with AxioCam HR color digital cameras (Carl Zeiss Microimaging) using 5×, 20×, 40× and 63× Plan-Neofluar and a 63× Plan/Apochromat objectives and the AxioVision 4.7 software package (Carl Zeiss Microimaging).

### Cresyl Violet Staining

Premounted sections are stained with cresyl violet for 30 s, differentiated in 70% ethanol, dehydrated by passing twice through 95% ethanol, 100% ethanol and xylene solutions, and mounted onto microscope slides with Eukitt® (Sigma).

### Evaluation of the Volume of the DARPP-32 Depleted Volume and Lobule V Volume

The extent of ataxin-3 lesions in the striatum or the cerebellar lobule V volume is analyzed by photographing, with a 1.25× objective, 8 DARPP-32 stained sections per animal (at 200 mm intervals) or 8 cerebellar cresyl violet sections per animal (at 210 mm intervals), selected so as to obtain complete sampling of the striatum or hemicerebellum, and by quantifying the area of the lesion or the lobule with a semiautomated image-analysis software package (Image J software, USA). The volume is then estimated with the following formula: volume=d(a₁+a₂+a₃ . . . ), where d is the distance between serial sections and a₁+a₂+a₃ are the areas for individual serial sections.

### Cell Counts and Morphometric Analysis of Ataxin-3 and Ubiquitin Inclusions, and Lobule V Molecular Layer

Coronal sections showing complete rostrocaudal sampling (1 of 11 sections) of the striatum are scanned with a 20× objective. The analyzed areas of the striatum encompass the entire region containing ATX-3 and ubiquitin inclusions, as revealed by staining with the anti-ataxin-3 and anti-ubiquitin antibodies. All inclusions are manually counted using a semiautomated image-analysis software package (Image J software, USA). Inclusions diameter is assessed by scanning the area above the needle tract in four different sections, using a 63× objective. At least 100 inclusions per animal are analyzed using LSM Image Browser. Inclusions diameter is further assessed by double staining of ataxin-3 inclusions with cleaved caspase-3 by scanning the area above the needle tract in three different sections, using a 63× objective. At least 100 inclusions per animal are analyzed using LSM Image Browser.

Midsagittal sections of hemicerebellum are scanned with a 20× objective. All calbindin-positive Purkinje cells of lobule V of six sections at 210 mm intervals are manually counted. Lobule V molecular layer thickness is assessed by the mean of four different measures. In each image, a boundary line around the molecular layer from, but excluding, the Purkinje cell bodies to the pial surface was drawn by hand using a semiautomated image-analysis software package (Image J software, USA).

### Western-Blot Analysis

For assessment of ataxin-3 proteolysis in the lentiviral model of Machado-Joseph disease, transcardial perfusion of the mice is performed with ice-cold phosphate buffered saline containing 10 mM ethylenediaminetetraacetic acid (EDTA) and 10 mM of the alkylating reagent N-ethylmaleimide, to avoid post-mortem calpain overactivation. The injected striata are then dissected and immediately sonicated in radioimmunoprecipitation assay buffer (RIPA) buffer (50 mM Tris-HCl, pH 7.4, 150 mM NaCl, 7 mM EDTA, 1% NP-40, 0.1% sodium dodecyl sulphate (SDS), 10 mg/ml dithiothreitol (DTT), 1 mM phenylmethylsulphonyl fluoride (PMSF), 200 mg/ml leupeptin, protease inhibitors cocktail).

One week before cell lysis, cerebellar granule neurons are treated with 200 mM N-methyl-D-aspartate (NMDA) plus 2.5 mM CaCl₂) in Krebs buffer without MgCl₂ for 1 h for excitatory stimulation and subsequently cultured in fresh medium plus inhibitor or DMSO until harvest and sonication in RIPA buffer, as described above.

Equal amounts (20 mg of protein) are resolved on 12% SDS-polyacrylamide gels and transferred onto polyvinylidene difluoride (PVDF) membranes. Immunoblotting is performed using the monoclonal anti-ataxin-3 antibody (1H9, 1:1000; Chemicon, Temecula, Calif.), monoclonal anti-polyglutamine antibody (1C2, MAB1574, 1:1000; Chemicon), monoclonal anti-myc tag (clone 4A6, 1:1000; Cell Signaling), monoclonal anti-spectrin antibody (MAB1622, 1:1000; Chemicon) and monoclonal anti-β-actin (clone AC-74, 1:5000; Sigma) or monoclonal anti-p-tubulin I (clone SAP.4G5, 1:15000; Sigma). Semiquantitative analysis is carried out using Quantity-one 1-D image analysis software version 4.5. A partition ratio with actin or tubulin is calculated.

Purification of Total RNA from Striata of Mice and cDNA Synthesis

Mice are sacrificed by cervical dislocation and injected striata are dissected and stored overnight at 4° C. in tubes containing RNAlater RNA stabilization reagent (QIAGEN). Samples are then kept at −80° C. until extraction of RNA. Total RNA is isolated using the RNeasyMiniKit (QIAGEN) according to the manufacturer's instructions. Briefly, after cell lysis, the total RNA is adsorbed to a silica membrane, washed with the recommended buffers and eluted with 30 ml of RNase-free water by centrifugation. Total amount of RNA is quantified by optical density (OD) using a Nanodrop 2000 Spectrophotometer (Thermo Scientific) and the purity is evaluated by measuring the ratio of OD at 260 and 280 nm. Complementary DNA (cDNA) is then obtained by conversion of 1 mg of total RNA using the iScript Select cDNA Synthesis Kit (Bio-Rad) according to the manufacturer's instructions and stored at −80° C.

Quantitative Real-Time Polymerase Chain Reaction (qRT-PCR)

Quantitative PCR is performed in an iQ5 thermocycler (Bio-Rad) using 96-well microtitre plates and the QuantiTect SYBR Green PCR Master Mix (QIAGEN). The primers for the target human gene (ATXN3, NM_004993) and the reference mouse genes (Hprt, NM_013556 and Gapdh, NM_008084) are pre-designed and validated by QIAGEN (QuantiTect Primers, QIAGEN). A master mix is prepared for each primer set containing the appropriate volume of QuantiTect SYBR Green PCR Master Mix (QIAGEN), QuantiTect Primers (QIAGEN) and template cDNA. All reactions are performed in duplicate and according to the manufacturer's recommendations: 95° C. for 15 min, followed by 40 cycles at 94° C. for 15 s, 55° C. for 30 s and 72° C. for 30 s. The amplification efficiency for each primer pair and the threshold values for threshold cycle determination (Ct) are determined automatically by the iQ5 Optical System Software (Bio-Rad). The mRNA fold increase or fold decrease with respect to control samples is determined by the Pfaffl method, taking into consideration different amplification efficiencies of all genes.

### Statistical Analysis

Statistical analysis is performed using unpaired Student's t-test or one-way ANOVA followed by Bonferroni test for selected pairs comparison. Values of P≤0.05 are considered statistically significant; P<0.01 very significant; and P<0.001 extremely significant.

Although the present disclosure has been described with reference to embodiments and examples, it should be understood that numerous and various modifications can be made without departing from the spirit of the present disclosure. Accordingly, the present disclosure is limited only by the following claims.

All references cited herein, including patents, patent applications, papers, text books, and the like, and the references cited herein, to the extent that they are not already, are hereby incorporated by reference in their entirety. In the event that one or more of the incorporated literature and similar materials differ from or contradict this application, including but not limited to defined terms, term usage, described techniques, or the like, this application controls.

What is claimed is:

1. A method of treating a neurological disease or disorder associated with protein aggregation, the method comprising administering to a subject in need thereof a compound of Formula (I): 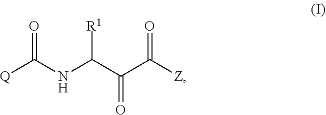
or a pharmaceutically acceptable salt or prodrug thereof, wherein:
R¹ is —C₁₋₆alkyl or —(CH₂)ₙ—C₆₋₁₀aryl optionally substituted with one, two, or three substituents independently selected from the group consisting of: -halo, —C₁₋₆ alkyl, —C₁₋₆haloalkyl, —C₁₋₆alkoxy, and —C₁₋₆haloalkoxy;
Z is —NR²R³ or —OR⁴;
R² is -hydrogen or —C₁₋₆ alkyl;
R³ is -hydrogen, —C₁₋₆ alkyl, —C₃₋₁₀ cycloalkyl, or —OR⁴;
R⁴ is -hydrogen or —C₁₋₆ alkyl;
Q is -5-10-membered heteroaryl optionally substituted with one, two, or three substituents independently selected from the group consisting of: -halo; —C₁₋₆ alkyl; —C₁₋₆haloalkyl; —C₁₋₆ alkoxy; —C₁₋₆ haloalkoxy; and —C₆₋₁₀ aryl optionally substituted with one, two, or three substituents independently selected from the group consisting of: -halo, —C₁₋₆ alkyl, —C₁₋₆ haloalkyl, —C₁₋₆alkoxy, and —C₁₋₆haloalkoxy; or
Q is —C₆₋₁₀aryl optionally substituted with one, two, or three substituents independently selected from the group consisting of: -halo; —C₁₋₆ alkyl; —C₁₋₆ haloalkyl; —C₁₋₆alkoxy; —C₁₋₆haloalkoxy; and -5-10-membered heteroaryl optionally substituted with one, two, or three substituents independently selected from the group consisting of: -halo, —C₁₋₆ alkyl, —C₁₋₆ haloalkyl, —C₁₋₆alkoxy, and —C₁₋₆haloalkoxy; and
n is 1 or 2.

2. The method of claim 1, wherein the compound is a compound of Formula (1-a) 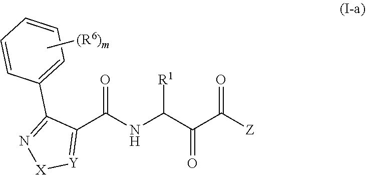
wherein:
X is S or NR⁵;
Y is CH or N;
R⁵ is -hydrogen or —C₁₋₆ alkyl;
each R⁶ is independently selected from the group consisting of: -halo, —C₁₋₆ alkyl, —C₁₋₆ haloalkyl, —C₁₋₆ alkoxy, and —C₁₋₆ haloalkoxy; and
m is 0, 1, 2, or 3.

3. The method of claim 2, wherein X is S.

4. The method of claim 2, wherein X is NR⁵.

5. The method of claim 4, wherein R⁵ is hydrogen or —CH₃.

6. The method of any one of claims 2 to 5, wherein Y is CH.

7. The method of any one of claims 2 to 5, wherein Y is N.

8. The method of any one of claims 2 to 7, wherein each R⁶ is independently —F, —Cl, —CH₃, —CF₃, —OCH₃, or —OCF₃.

9. The method of any one of claims 2 to 8, wherein m is 0.

10. The method of any one of claims 2 to 8, wherein m is 1.

11. The method of any one of claims 2 to 8, wherein m is 2.

12. The method of any one of claims 2 to 8, wherein m is 3.

13. The method of claim 1, wherein the compound is a compound of Formula (1-b) 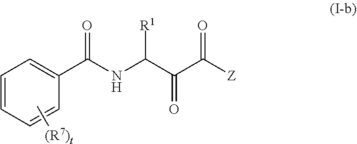
wherein:
each R⁷ is independently selected from the group consisting of: -halo, —C₁₋₆ alkyl, —C₁₋₆ haloalkyl, —C₁₋₆ alkoxy, and —C₁₋₆ haloalkoxy; and
t is 0, 1, 2, or 3.

14. The method of claim 13, wherein each R⁷ is independently —F, —Cl, —CH₃, —CF₃, —OCH₃, or —OCF₃.

15. The method of claim 13 or 14, wherein t is 0.

16. The method of claim 13 or 14, wherein t is 1.

17. The method of claim 13 or 14, wherein t is 2.

18. The method of claim 13 or 14, wherein t is 3.

19. The method of any one of claims 1 to 18, wherein Z is —NR²R³.

20. The method of any one of claims 1 to 19, wherein R² is -hydrogen.

21. The method of any one of claims 1 to 20, wherein R³ is -hydrogen, —CH₃, or -cyclopropyl.

22. The method of any one of claims 1 to 20, wherein R³ is —OH or —OCH₃.

23. The method of any of claims 1 to 18, wherein Z is —OR⁴.

24. The method of claim 23, wherein R⁴ is -hydrogen or —CH₃.

25. A method of treating a neurological disease or disorder associated with protein aggregation, the method comprising administering to a subject in need thereof a compound selected from the group consisting of: 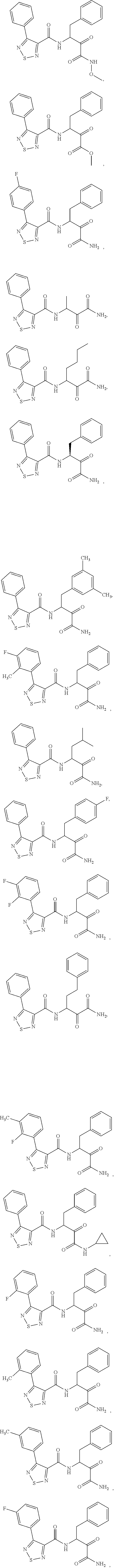 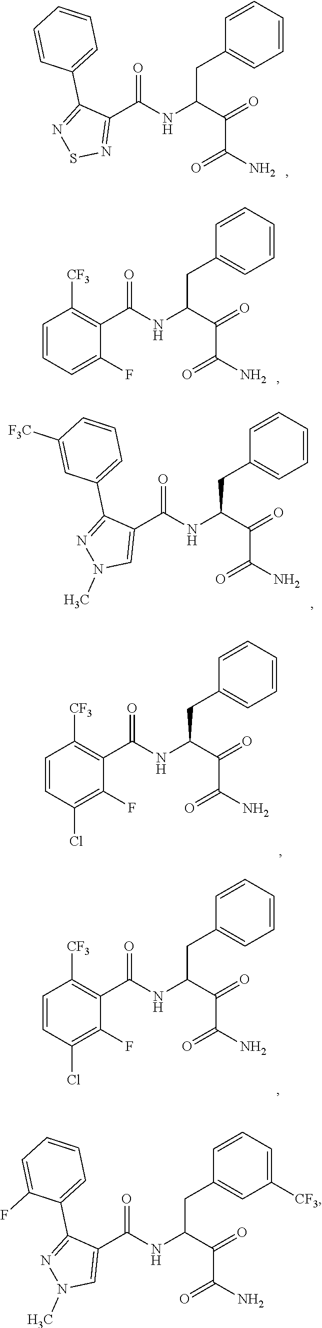
and pharmaceutically acceptable salts thereof.

26. The method of any one of claims 1 to 25, the method further comprising administering to the subject one or more second pharmaceutical agents.

27. The method of claim 26, wherein the second pharmaceutical agent is selected from tetrabenazinem, deutetrabenazine, citalopram, escitalipram, fluoxetine, sertraline, quetiapine, risperidone, haloperidol, chlorpromazine, valproate, carbamazepine, lamotrigine, levodopa, baclofen, and botulinum toxin.

28. The method of any one of claims 1 to 27, wherein the neurological disorder associated with protein aggregation is a polyglutamine disease or disorder.

29. The method of claim 28, wherein the polyglutamine disorder is Huntington's disease, Machado-Joseph disease, dentatorubral-pallidoluysian atrophy, spinal and bulbar muscular atrophy, spinocerebellar ataxia type 1 (SCA1), spinocerebellar ataxia type 2 (SCA2), spinocerebellar ataxia type 6 (SCA6), spinocerebellar ataxia type 7 (SCA7), or spinocerebellar ataxia type 17 (SCA17).

30. The method of claim 28, wherein the neurological disorder associated with protein aggregation is Alzheimer's disease, Parkinson's disease, or amyotrophic lateral sclerosis.
